(19) United States
(12) Patent Application Publication
(10) Pub. No.: US 2010/0241358 A1
(43) Pub. Date: Sep. 23, 2010

(54) ANALYSIS

(75) Inventors: Frederic Zenhausern, Tempe, AZ (US); Ralf Lenigk, Tempe, AZ (US); Andrew Hopwood, Solihull (GB); Pieris Koumi, Solihull (GB)

Correspondence Address: MERCHANT & GOULD PC, P.O. BOX 2903, MINNEAPOLIS, MN, 55402-0903, US

(73) Assignees: Forensic Science Service Limited, Birmingham (GB); Arizona Board of Regents for and on behalf of Arizona State University, Scottsdale, AZ (US)

(21) Appl. No.: 12/558,244

(22) Filed: Sep. 11, 2009

Related U.S. Application Data
(60) Provisional application No. 61/096,424, filed on Sep. 12, 2008.

(51) Int. Cl. G06F 19/00; G01N 33/48

(52) U.S. Cl. 702/19

(57) ABSTRACT

A method of verifying the performance of a size standard, formed of a plurality of size standard elements, used in a nucleic acid analysis method is provided. The verification is achieved by introducing the size standard to an analysis stage and performing a size based separation on the size standard. The size based separation defines an experimental position for each size standard element. A comparison is made as to whether or not the experimental position for the size standard element corresponds with expected position. The comparison allows a decision to be made on whether or not to use the size standard, which is dependent upon whether or not the experimental position of the size standard element corresponds with the expected position.

This application claims benefit of Ser. No. 61/096424, filed Sep. 12, 2008 in the United States and which application(s) are incorporated herein by reference. A claim of priority to all, to the extent appropriate is made.

This invention is concerned with improvements in and relating to analysis, particularly analysis of samples containing nucleic acids.

To improve the speed with which nucleic acid analysis results are available for consideration, there is a drive towards the use of analysis devices in the field, rather than in a centralised laboratory. This gives the desire to use a miniaturised device compared with device sizes encountered in laboratories.

In providing a miniaturised device, the number of capillaries available for analysis is reduced. This impacts upon the ability to use a channel for a size standard separate from a channel used for analysis of the sample.

Amongst the potential aims of the invention may be to provide a simplified analysis approach. Amongst the potential aims of the invention may be to provide an analysis approach in which the only size standard is in the capillary used for the analysis of the sample. Amongst the potential aims of the invention may be to provide a single capillary based analysis approach.

According to a first aspect of the invention we provide a method of verifying the performance of a size standard, the method comprising:
a) introducing a size standard to an analysis stage, the size standard including one or more size standard elements;
b) performing a size based separation on the size standard, the size based separation defining an experimental position for the size standard element for one or more size standard elements;
c) defining one or more characteristics for one or more size standard elements;
d) considering whether the experimental position for the size standard element corresponds with respect to one or more of the characteristics;
e) making a use of the size standard dependent upon whether or not the experimental position of the size standard element corresponds with respect to one or more characteristics.

The size standard may be of a standard or known in terms of one or more of its mass, number of base pairs, sequence, relative migration rate or speed.

The method may further comprise, preferably in respect of step c), determining a theoretical position for the same size standard element. The method may further comprise, preferably in respect of step d) considering whether the experimental position and theoretical position for a given size standard element fit to one another. The method may further comprise, preferably in respect of step e) making a use of the size standard dependant upon whether or not the experimental position and theoretical position for the given size standard element fit to one another.

The method may further comprise, preferably in respect of step a), introducing a size standard to an analysis stage, the size standard including three or more size standard elements. The method may further comprise, preferably in respect of step b), performing a size based separation on the size standard, the size based separation defining a size standard element position for three or more size standard elements. The method may further comprise selecting three or more size standard elements. The method may further comprise, preferably in respect of step c), defining a relationship between the size standard element position and size standard element size for each of the selected size standard elements. The method may further comprise, preferably in respect of step d), checking whether the relationship has a pre-determined form, preferably a linear relationship. The method may further comprise, preferably in respect of step e) deciding not to make use of the size standard if the relationship is not a linear relationship.

The method may further comprise, preferably in respect of step a) introducing a size standard to an analysis stage, the size standard including three or more size standard elements. The method may further comprise, preferably in respect of step b), performing a size based separation on the size standard, the size based separation defining a size standard element position for three or more size standard elements. The method may further comprise selecting two or more size standard elements. The method may further comprise, preferably in respect of step c), defining a relationship between the size standard element position and size standard element size, preferably, for each of the selected size standard elements. The method may further comprise, preferably in respect of step d), selecting a further size standard element. The method may further comprise, preferably in respect of step d), considering whether the size standard element position and size standard element size for the further size standard element fits the relationship. The method may further comprise, preferably in respect of step e), deciding not to make use of the size standard dependant if the size standard element position and size standard element size for the further size standard element does not fit the relationship.

The method may provide that step d is repeated for a plurality of different size standard elements. The method may provide that step d is repeated for each of the different size standard elements. The method may provide that step e) is repeated for a plurality of different size standard elements. The method may provide that step e) is repeated for each of the different size standard elements.

The method may provide that the considering of whether the experimental position corresponds with respect to one or more characteristics is repeated for a plurality of different size standard elements. The method may provide that the considering of whether the experimental position corresponds with respect to one or more characteristics is repeated for each of the different size standard elements. The method may provide that the making a use of the size standard dependent upon whether or not the experimental position of the size standard element corresponds with respect to one or more characteristics is repeated for a plurality of different size standard elements. The method may provide that the making a use of the size standard dependent upon whether or not the experimental position of the size standard element corresponds with respect to one or more characteristics is repeated for each of the different size standard elements.

The method may provide that the considering of whether the experimental position and theoretical position for a given size standard element fit to one another is repeated for a plurality of different size standard elements. The method may provide that the considering of whether the experimental position and theoretical position for a given size standard element fit to one another is repeated for each of the different size standard elements. The method may provide that the making a use of the size standard dependant upon whether or not the experimental position and theoretical position for the given size standard element fit to one another is repeated for a plurality of different size standard elements. The method may provide that the making a use of the size standard dependant upon whether or not the experimental position and theoretical position for the given size standard element fit to one another is repeated for each of the different size standard elements.

The method may provide that the defining a relationship between the size standard element position and size standard element size for each of the selected size standard elements is repeated for a plurality of different size standard elements. The method may provide that the defining a relationship between the size standard element position and size standard element size for each of the selected size standard elements is repeated for each of the different size standard elements. The method may provide that the checking of whether the relationship is a linear relationship may be repeated for a plurality of different size standard elements. The method may provide that the checking of whether the relationship is linear relationship may be repeated for each of the different size standard elements. The method may provide that the step of deciding not to make use of the size standard if the relationship is not a linear relationship may be repeated for a plurality of different size elements. The method may provide that the step of deciding not to make use of the size standard if the relationship is not a linear relationship may be repeated for each of the different size elements.

The method may provide that the considering whether the size standard element position and size standard element size for the further size standard element fits the relationship is repeated for a plurality of different size standard elements. The method may provide that the considering whether the size standard element position and size standard element size for the further size standard element fits the relationship is repeated for each of the plurality of different size standard elements. The method may provide that the step of deciding not to make use of the size standard dependant if the size standard element position and size standard element size for the further size standard element does not fit the relationship is repeated for a plurality of different size standard elements. The method may provide that the step of deciding not to make use of the size standard dependant if the size standard element position and size standard element size for the further size standard element does not fit the relationship is repeated for each of the plurality of different size standard elements.

The considering and/or checking, particularly of step d), may be made using a value and/or data classification and/or category. The considering and/or checking, particularly of step d), may be made using a range. The considering and/or checking may be deemed to correspond and/or fit and/or be a linear relationship where the value is within a range. The considering and/or checking may be deemed not to correspond and/or fit and/or be a linear relationship where the value is outside a range.

One or more size standard elements of size greater than the size standard element being considered and/or checked may be used, particularly in step d). One or more size standard elements of size less than the size standard element being considered and/or checked may be used, particularly in step d). Preferably at least one size standard of size greater than and at least one size standard of size less than the size standard being considered and/or checked is used, particularly in step d).

Preferably one of the one or more size standard elements of size greater than the size standard element being considered and/or checked which may be used, particularly in step d), is the next largest size standard element. Preferably one of the one or more size standard elements of size less than the size standard element being considered and/or checked which may be used, particularly in step d), is the next smallest size standard element.

Where the size standard element being considered and/or checked is the smallest size standard element in the size standard, the next two greater sized size standard elements may be used in the considering and/or checking. Where the size standard element being considered and/or checked is the largest size standard element in the size standard, the next two smallest sized size standard elements may be used in the considering and/or checking.

Preferably the method provides for the verification of the consistency of the size based separation, particularly the consistency of migration, particularly in respect of the whole, or at least a part. of the capillary or channel distance involved in the size based migration.

Preferably the size based separation on the size standard is performed in the same capillary or channel as the size based separation on a sample.

The first aspect of the invention may include any of the features, options or possibilities set out within this document, including in the other aspects.
According to a second aspect of the invention we provide a method of analysing a sample, the sample including one or more elements, the method comprising:
introducing a sample to an analysis stage;
performing a size based separation on the sample, the size based separation resulting in a position for each of the one or more elements;
introducing a size standard to an analysis stage, the size standard including three or more size standard elements;
performing a size based separation on the size standard, the size based separation resulting in a size standard element position for three or more size standard elements;
comparing the position of at least one element with the position of a size standard element;
defining a characteristic of the element from the comparison of positions, wherein, the method of analysis including a method of verifying the performance of a size standard including the steps of:
c) defining one or more characteristics for one or more size standard elements;
d) considering whether the experimental position for the size standard element corresponds with respect to one or more of the characteristics;
e) making a use of the size standard dependent upon whether or not the experimental position of the size standard element corresponds with respect to one or more characteristics.

The size standard may be of a standard or known in terms of one or more of its mass, number of base pairs, sequence, relative migration rate or speed.

The position may be considered as a position along a capillary or channel. The position may be considered as a distance relative to a reference point. The position may be considered as a spatial characteristic and/or frequency characteristic.

The one or more characteristics may be the position and/or a physio-chemical property and/or value and/or range.

The method of verifying may provide for:
a) selecting two or more size standard elements;
b) defining a relationship between the size standard element position and size standard element size for each of the selected size standard elements;
c) selecting a further size standard element;
d) considering whether the size standard element position and size standard element size for the further size standard element fits the relationship;
e) making a use of the size standard dependant upon whether or not the size standard element position and size standard element size for the further size standard element fits the relationship.

Preferably the size based separation on the size standard is performed in the same capillary or channel as the size based separation on a sample.

Preferably the comparing of the position of at least one element with the position of a size standard element is made direct. Preferably the comparing of the position of at least one element with the position of a size standard element is made by correlation. Preferably the comparing of the position of at least one element with the position of a size standard element is made by measuring the position of the element relative to one or more size standard elements.

The method may be a method of analysing nucleic acids within a sample. The one or more elements may be nucleic acids. The method may be a method of analysing DNA within a sample. The one or more elements may be elements of DNA. The method may be a method of analysing alleles, particularly short tandem repeats, and/or single nucleotide polymorphisms (SNPs) or other biomarker, within a sample. The one or more elements may be alleles, particularly short tandem repeats. The method may be a method of analysing multiple loci within a sample. The one or more elements may be one or more loci.

The method may include the determination of the identity of the one or more elements. The determination may provide an allelic profile for the sample. The determination may provide a profile and/or a genotype for the sample.

The method may include the generation of a result. The method may include the storage of a result. The method may include exporting a result. The method may include displaying a result. The result may be the identity of one or more elements and/or the allele present for one or more elements and/or an allele profile and/or a genotype. The method may be computer implemented.

The size based separation may be provided by electrophoresis. The size based separation may be provided within a polymer matrix, such as a gel, hydrogel, aerogel or hybrid mixture of inorganic and organic materials, preferably that can form a porous medium. The size based separation may be provided within a fluid, such as a free solution conditioned for migrating size standards or their derivatives. The size based separation may be provided within a confined environment, such as a channel or capillary.

The position for each of the one or more elements may be a distance allow a direction of travel. The position may be a distance from the location to which the sample is introduced.

Preferably the size based separation for the sample and for the size standard are provided by the same electrophoresis. Preferably the size based separation for the sample and the size standard are provided in the same separation medium, such as a gel. Preferably the size based separation for the sample and the size standard are provided in the same capillary.

The size standard may include, preferably about, five or more size standard elements. The size standard may include, preferably about, eight or more size standard elements.

The second aspect of the invention may include any of the features, options or possibilities set out within this document, including in the other aspects

According to a third aspect of the invention we provide a device for analysing a sample, the device comprising:
a confined environment, preferably a channel, for introducing a size standard including one or more size standard elements;
a size based separation stage;
a position detector for generating an experimental position for one or more of the size standard elements;
a data processor, the data processor including:
   an input for experimental positions for one or more size standard elements;
   memory or logic for defining one or more characteristics for one or more size standard elements;
      a comparator for considering whether the experimental position for the size standard element corresponds with respect to one or more of the characteristics.

The apparatus may provide a data processor including:
memory or logic for defining a relationship between the size standard element position and size standard element size for each of the selected size standard elements
an input for size standard element position in respect of a further size standard element;
a comparator for calculating whether the size standard element position and size standard element size for the further size standard element fits the relationship.

The third aspect of the invention may include any of the features, options or possibilities set out within this document, including in the other aspects.

Various embodiments of the invention will now be described, by way of example only.

The nucleic acid analysis process frequently involves a sample preparation stage, sample amplification stage and sample analysis stage. In the sample analysis stage there is a need to identified the characteristics of interest of the sample. These characteristics are then reported to the user.

One characterisation approach considers the particular identities present at one or more identities capable of variation. An example of such an approach is the consideration of allele identities at locations, loci, known to be variable. The alleles vary in identity due to variations in their size. The variations in size are caused by variations in the form of the short tandem repeat, STR, sequence at these loci. Typically, the STR sequence is formed of a series of 4 base repeats, with varying numbers of each of the repeats. However, other variations aside from the number and type of 4 base repeats present may occur. The variation in the number of repeat units present and/or other bases present gives rise to different size alleles.

In the context of forensic science, the consideration of the variation in allele identity between loci, and with many loci being considered, enables useful information about the biological species, in particular an individual who was the source of the sample to be determined; their genotype. The selection of the loci is made so as to give sufficient variability between individuals to make the genotype distinctive. For example, a person's genotype can be compared with the characteristics revealed from another sample to form a link between the person and the another sample or to dismiss any such link.

There is an increasing drive towards miniaturisation in the context of methods and devices to analysis samples containing nucleic acids, including samples containing DNA. Miniaturisation allows for the analysis to be performed using a device which can be brought to the location the sample is collected from; as opposed to conveying the sample to a remote laboratory. In the context of forensic science, and particularly in the case of human identification, this enables analysis to be performed at, or in close proximity to a crime scene from which a sample is recovered, for instance. In medical contexts, it allows analysis on the hospital ward or doctor's surgery. Further applications include tissue bio-banking, where tissue samples could be processed and according to the disclosed methodology sample tracking and/or monitoring capabilities could be provided. As well as convenience, the result is obtained far faster using such an approach.

Miniaturisation, however, brings constraints on the methods and apparatus used.

In a conventional sample analysis stage, the sample will be analysed in one or more capillaries or lanes of an electrophoresis device, with for instance 32 or 64 capillaries or channels being processed at once.

In a miniaturised device, however, a single capillary or channel is often desirable for the sake of size and the simplicity of sample preparation; generally collection and introduction stages. This presents requirements with being able to accurately analyse the sample and the identity of the characteristics.

In conventional analysis stages, using a large number of capillaries or channels, two different sorts of size standards are used to ensure accurate sizing.

Firstly, in a capillary or channel adjacent to the capillary or channel to be used for an unknown sample, one whose characteristics are to be determined, an allelic ladder is analysed.

An allelic ladder includes a series of alleles for each of the series of loci of interest. For a locus, the series of alleles varies from one another according to the variation in the STR sequence to be found between individuals. Thus, for example, alleles with 6, 7, 8 up to 18 lots of the 4 base repeat varying sequence may be provided for a locus. Other sets of alleles are provided for other loci. The alleles correspond in sequence and size to alleles commonly expected to occur in the unknown sample.

As electrophoresis occurs, the alleles move different distances according to their size and hence relative mobility. The positions of the alleles of the allelic ladder are revealed by the dyes or other labels associated with them; with a different dye or label for each locus that has potentially overlapping alleles. The equivalent dye or label is used in conjunction with the allele(s) from the equivalent locus in the unknown sample. The alleles of the allelic ladder can be compared with the alleles of the unknown sample to identify which alleles are present in the unknown sample. Comparison of position based upon allelic ladder allele to unknown allele comparison is desirable because the alleles of the allelic ladder and unknown sample are the same (in terms of sequence, size and label) and hence can be expected to behave equivalently to one another.

Secondly, in the same capillary or channel to that in which the unknown sample is considered, an internal lane standard is provided. The internal lane standard is a set of oligonucleotides of precisely known sizes. These are unrelated and quite different from the alleles in terms of their sequence. Again, dyes or other labels are associated with the oligonucleotides to reveal their position. The dyes or labels differ from those used in the case of the allelic ladder and unknown sample. In this way, when electrophoresis is performed, the internal lane standard gives rise to a series of regularly spaced markings which are distinctly coloured compared with any alleles.

The second, internal lane standard, is needed because there is the possibility that the capillary or channel in which the allelic ladder is provided will differ in properties compared with the capillary or channel in which the unknown is provided. This would result in different distances being covered by the same allele in the two capillaries or channels and hence give a false result. The use of the internal lane standard means it is possible to cross-verify the performance of the capillary or channel with the allelic ladder and the capillary or channel with the unknown sample.

Thus the analysis of the positions uses the internal lane standard in a comparison process with the alleles of the allelic ladder in the adjacent capillary or channel to check the relative performance of the two capillaries or channels. An adjustment is then applied. The alleles of the allelic ladder are then compared with the unknown alleles in the adjacent channel to reveal the size of the unknown alleles by virtue of their position.

There is no direct use or comparison of the internal lane standard with the alleles of the unknown sample; the alleles in the same lane as the internal lane standard.

This approach means that two capillaries or channels are needed to process a single unknown sample. This increases the size of the unit and increases the complexity by providing two separate capillaries or channels for which equivalent material introduction and equivalent electrophoresis need to be provided.

In the present invention, the alleles of an unknown sample are characterised using only a single capillary or channel.

The single capillary or channel is used to provide a size based separation of the unknown sample into its indicative alleles for the various loci. The position of those alleles is revealed by the associated dyes or other labels.

The same single capillary or channels is also used to separate, simultaneously, the internal lane standard into its component parts. The positions of the component parts are revealed by the different associated dyes or other labels used for the internal lane standard.

Before making the allocation of size determination for the unknown sample, the consistency of migration within the single capillary or channel is checked.

This is done by establishing the experimental position for each of the oligonucleotides in the internal lane standard compared with the theoretical position expected because of its known size.

In more detail, a single oligonucleotide from within the internal lane standard is selected for checking. It is in effect treated as an oligonucleotide of unknown size. The adjacent oligonucleotide of lower size and the adjacent and next oligonucleotides of larger size are selected for use in the checking process. For example, a plot of size against log of mobility (position) is made using these three oligonucleotides. The plot is expected to reveal a linear relationship, preferably with a straight line, and any deviation detected relative to that implies inconsistent migration. Furthermore, the single oligonucleotide being checked should lie on that straight lie too. Again a deviation relative to that position implies inconsistent migration. Finally, the position of that oligonucleotide on the line indicates its experimental size. This experimental size can be compared with the theoretical size known from the construction of the internal lane standard. Again any deviation between the theoretical size and the experimental size indicates inconsistent migration.

This process is repeated for each of the oligonucleotides of the internal lane standard in turn.

With respect to the lower most oligonucleotide, then the two or three oligonucleotides adjacent in size can be used. Similarly, for the largest oligonucleotide, the two or three oligonucleotides which are smaller and adjacent can be used.

The adjacent oligonucleotide of greater size and the adjacent and next oligonucleotides of smaller size can equally well be selected for use in the checking process.

As a result of this process the consistency of migration across the whole of the capillary or channel distance is checked and as a result, the consistency of migration throughout the entire range of alleles likely to be encountered in the unknown sample. The checking process is also performed in detail for each part; not just an overall impression for the whole of the capillary or channel.

Once the consistency of the migration has been checked, the allocation of a size, and hence an identity to the alleles is made by comparing the unknown alleles with the internal lane standard in a similar manner. The adjacent oligonucleotides of the internal lane standard are used to define the plot relative to two known sizes and the position of the allele of the unknown sample on that plot determines it size.

Whilst the above mentioned approach is particularly useful in single capillary or channel scenarios, the ability to analyse an unknown sample using a single capillary or channel is also beneficial in multiple capillary or channel devices. Because there is no longer a need to provide the allele ladder in one or more of the capillaries or channels (frequently quite a few of the capillaries or channels), those channels are also available for processing unknowns. This gives increased capacity to the apparatus and hence reduces unit cost for the processing.

1. A method of verifying the performance of a size standard for potential use in a nucleic acid analysis method, the method of verifying comprising:
a) introducing a size standard to an analysis stage, the size standard including one or more size standard elements;
b) performing a size based separation on the size standard, the size based separation defining an experimental position for the size standard element for one or more size standard elements;
c) defining one or more characteristics for one or more size standard elements;
d) considering whether the experimental position for the size standard element corresponds with respect to one or more of the characteristics;
e) making a use of the size standard dependent upon whether or not the experimental position of the size standard element corresponds with respect to one or more characteristics.

2. A method according to claim 1, wherein the method further comprises:
in respect of step c), determining a theoretical position for a size standard element of the same size as a size standard element for which an experimental position has been defined;
in respect of step d) considering whether the experimental position and theoretical position for a given size standard element fit to one another; and
in respect of step e) making a use of the size standard dependant upon whether or not the experimental position and theoretical position for the given size standard element fit to one another.

3. A method according to claim 2, wherein the use made of the size standard is not to use that size standard in a nucleic acid analysis method, wherein the experimental position and theoretical position for the given size standard element do not fit to one another.

4. A method according to claim 1, wherein the method further comprises:
in respect of step c), defining a relationship between the size standard element position and size standard element size for each of three or more selected size standard elements;
in respect of step d), preferably checking whether the relationship is a linear relationship;
in respect of step e), making a use of the size standard dependant upon whether or not the relationship is a linear relationship.

5. A method according to claim 4, wherein the use made of the size standard is not to use that size standard in a nucleic acid analysis method, where the relationship is not a linear relationship.

6. A method according to claim 5, wherein the relationship is an equation to link the size standard element position and size standard element size for each of three or more selected size standard elements when preferably expressed as a plot of size against log mobility.

7. A method according to claim 1, wherein the method further comprises:
in respect of step c), defining a relationship between the size standard element position and size standard element size for each of three or more selected size standard elements;
in respect of step d), selecting a further size standard element and considering whether the size standard element position and size standard element size for the further size standard element fits the relationship; and
in respect of step e), making a use of the size standard dependant upon whether or not the size standard element position and size standard element size for the further size standard element fits the relationship.

8. A method according to claim 7, wherein the use made of the size standard is not to use that size standard in a nucleic acid analysis method, where the size standard element position and size standard element size for the further size standard element does not fit the relationship.

9. A method according to claim 1, wherein step d) is performed in respect of the experimental position of each of the size standard elements.

10. A method according to claim 2, wherein step d) is performed in respect of the experimental position of each of the size standard elements.

11. A method according to claim 4, wherein step d) is performed in respect of the experimental position of each of the size standard elements.

12. A method according to claim 7, wherein step d) is performed in respect of the experimental position of each of the size standard elements.

13. A method of analysing a nucleic acid containing sample, the sample including one or more elements, the method comprising:
introducing a sample to an analysis stage;
performing a size based separation on the sample, the size based separation resulting in a position for each of the one or more elements;
introducing a size standard to an analysis stage, the size standard including three or more size standard elements;
performing a size based separation on the size standard, the size based separation resulting in a size standard element position for three or more size standard elements;
comparing the position of at least one element with the position of a size standard element;
defining a characteristic of the element from the comparison of positions, wherein, the method of analysis including a method of verifying the performance of a size standard including the steps of:
c) defining one or more characteristics for one or more size standard elements;
d) considering whether the experimental position for the size standard element corresponds with respect to one or more of the characteristics;
e) making a use of the size standard dependent upon whether or not the experimental position of the size standard element corresponds with respect to one or more characteristics.

14. A device for analysing a nucleic acid containing sample, the device comprising:
a channel for introducing a size standard including one or more size standard elements;
a size based separation stage;
a position detector for generating an experimental position for one or more of the size standard elements;
a data processor, the data processor including:
an input for experimental positions for one or more size standard elements;
memory or logic for defining one or more characteristics for one or more size standard elements;
a comparator for considering whether the experimental position for the size standard element corresponds with respect to one or more of the characteristics.

15. A device according to claim 14, wherein said channel is made of a substrate material or materials selected from one or more of: glass, silicon, polymeric materials or other plastics materials

16. A device according to claim 15, wherein the polymeric material is or includes polycarbonate, polyethylene or cyclic olefinic.

17. A device according to claim 14, wherein the device is at least impart produced by hot embossing, injection moulding, imprinting processing, nanoimprinting lithography or conventional semi-conductor processing.

18. A device according to claim 14, comprising embedded digital memory circuitry.
